(19) United States
(12) Patent Application Publication
(10) Pub. No.: US 2022/0415508 A1
(43) Pub. Date: Dec. 29, 2022

(54) PREDICTION DEVICE

(71) Applicant: MIRAI SCIEN CO. LTD., Nagoya-shi, Aichi (JP)

(72) Inventor: Tomofumi HATAKEYAMA, Aichi (JP)

(73) Assignee: MIRAI SCIEN CO. LTD., Nagoya-shi, Aichi (JP)

(21) Appl. No.: 17/780,738

(22) PCT Filed: Nov. 25, 2020
(86) PCT No.: PCT/JP2020/043798

§ 371 Date: May 27, 2022

(30) Foreign Application Priority Data
Dec. 17, 2019 (JP) 2019-227555

(51) Int. Cl. G16H 50/20; G16H 30/20

(52) U.S. Cl. CPC G16H 50/20; G16H 30/20

(57) ABSTRACT

A prediction server includes: a data save unit that acquires diagnosis data from a diagnosis server; a training unit that constructs a prediction model that predicts a possibility of occurrence of the disorder in an arbitrary area and on an arbitrary date by training a learning model about a correlation among an area indicated by first position information, a date indicated by a diagnosis date, and a type of disorder in a machine learning manner, in which the type of the disorder is a diagnosis result estimated by the diagnosis server based on a diseased portion image by using a diagnosis model trained about a correlation between the diseased portion image and the disorder in the machine learning manner.

### TECHNICAL FIELD

The present invention relates to a technique for predicting a possibility of occurrence of a disorder on a plant.

### BACKGROUND ART

In the related art, a technique for predicting a possibility of occurrence of disease and insect pest on a plant is known. For example, Patent Literature 1 and Patent Literature 2 disclose a technique for predicting types of disease and insect pest occurring in a crop based on a type of cultivation crop, weather information of a cultivation area, a weather condition under which the disease and insect pest occur.

### CITATION LIST

### Patent Literature

Patent Literature 1: JP 2006-115704 A

Patent Literature 2: JP 2016-167214 A

### SUMMARY OF INVENTION

### Problems to be Solved by the Invention

However, in the above-described related art, the occurrence of disease and insect pest is only predicted based on empirically estimated conditions, and an actual occurrence status of the disease and insect pest in the past is not considered. An aspect of the present invention has been made in view of the above problems, and an object of the present invention is to accurately predict a possibility of occurrence of at least one of disease and insect pest or a physiological disorder occurring on a plant.

### Means for Solving the Problems

In order to solve the above problem, a prediction device according to an aspect of the present invention includes: a data acquisition unit that acquires, from a diagnosis device that diagnoses a disorder occurring on a plant, first position information indicating a growth area of the plant, a diagnosis date, and a type of the disorder; and a training unit that constructs a prediction model predicting a possibility of occurrence of the disorder in an arbitrary area and on an arbitrary date by training a learning model about a correlation among an area indicated by the first position information, a date indicated by the diagnosis date, and a type of the disorder in a machine learning manner, and the type of the disorder is a diagnosis result estimated by the diagnosis device based on a diseased portion image of the plant on which the disorder occurs by using a diagnosis model trained about a correlation between diseased portion images of the plant on which the disorder occurs and the type of the disorder in the machine learning manner.

### Advantageous Effects of Invention

According to the aspect of the present invention, a possibility of occurrence of at least one of disease and insect pest or a physiological disorder occurring on a plant can be accurately predicted.

### BRIEF DESCRIPTION OF DRAWINGS

FIG. 1 is a diagram illustrating an overview of various systems according to a first embodiment.

FIG. 2 is a block diagram illustrating a configuration of main parts of the various systems.

FIG. 3 is a sequence diagram illustrating a flow of a model construction process.

FIG. 4 is a sequence diagram illustrating a flow of a prediction process.

FIG. 5 is a diagram illustrating an example of a display screen showing a prediction result.

FIG. 6 is a block diagram illustrating a configuration of main parts of various systems according to a second embodiment.

FIG. 7 is a block diagram illustrating a configuration of main parts of various systems according to a fourth embodiment.

### MODES FOR CARRYING OUT THE INVENTION

A plant disorder prediction system according to the present invention is a system that predicts a possibility of occurrence of a disorder on a plant. Note that in the present invention, the term “disorder” indicates at least one of a disease, an insect pest, or a physiological disorder occurring on a plant. Furthermore, in the present invention, the term “type of disorder” indicates a general name of disease and insect pest or a physiological disorder, such as powdery mildew, an aphid, or a drying disorder. The plant disorder prediction system according to the present invention cooperates with a plant disorder diagnosis system. The plant disorder diagnosis system is a system that diagnoses a type of disorder occurring on a plant by using a captured image including a mutation portion (that is, the diseased portion) appearing on the plant due to the disorder. The plant disorder prediction system acquires data obtained when the plant disorder diagnosis system performs diagnosis and diagnosis result data, from the plant disorder diagnosis system. The plant disorder prediction system predicts a possibility of occurrence of any disorder on a plant growing in an arbitrary (predetermined) area on an arbitrary (predetermined) date by using a trained model created by using these data. A timing of the prediction and a method of setting the area and the date are not particularly limited. For example, the plant disorder prediction system may predict the occurrence probability of various disorders on the next day in each area in a prediction target area of the system once a day. Note that in the plant disorder prediction system and the plant disorder diagnosis system, a type of disorder and a type of plant, which are prediction targets, are not particularly limited. In the following embodiment, as an example, a case where the plant is a cultivation crop such as a vegetable, a fruit tree, or a flower will be described. Hereinafter, operation of the plant disorder diagnosis system and operation of the plant disorder prediction system according to the present invention will be described in detail based on the first embodiment to the third embodiment.

### First Embodiment

<<System Overview>>

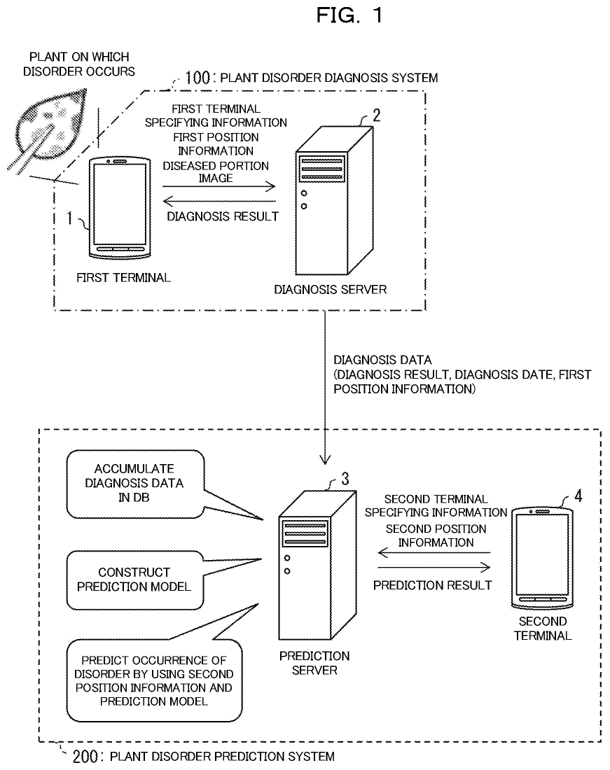

FIG. 1 is a diagram illustrating an overview of various systems (plant disorder diagnosis system 100 and plant disorder prediction system 200) according to the present embodiment. As illustrated in FIG. 1, the plant disorder diagnosis system 100 includes a first terminal 1 and a diagnosis server (diagnosis device) 2. Furthermore, the plant disorder prediction system 200 includes a prediction server (prediction device) 3 and a second terminal 4. In the present embodiment, the plant disorder diagnosis system 100 operates when the first terminal 1 executes a diagnosis application program (diagnosis application) installed in the first terminal 1. Furthermore, in the present embodiment, the plant disorder prediction system 200 operates when the second terminal 4 executes a prediction application program (prediction application) installed in the second terminal 4. Hereinafter, the application program is also simply referred to as an “application”.

In the following description, for convenience, a user who carries the first terminal 1 is referred to as a “first user”, and a user who carries the second terminal 4 is referred to as a “second user” for distinction. However, the first user and the second user may be the same person. Furthermore, information indicating a position of the first terminal 1 is referred to as “first position information”, and information indicating a position of the second terminal 4 is referred to as “second position information” for distinction. In addition, information for specifying the first terminal 1 is referred to as “first terminal specifying information”, and information for specifying the second terminal 4 is referred to as “second terminal specifying information” for distinction. However, a function of the first terminal 1 and a function of the second terminal 4 may be realized by one terminal device. That is, both the diagnosis application and the prediction application may be installed in the first terminal 1. Note that the first terminal specifying information and the second terminal specifying information may be, for example, identification numbers unique to each terminal. Furthermore, a plurality of the first terminals 1 may be present in the plant disorder diagnosis system 100, and a plurality of the second terminals 4 may be present in the plant disorder prediction system 200.

(Operation of Plant Disorder Diagnosis System 100)

The first terminal 1 executes processing to be described below according to the diagnosis application. First, the first terminal 1 prompts the user to capture an image including a diseased portion of the plant suspected of having a disorder. For example, the first terminal 1 displays an operation guide or the like for the user on a display surface of the first terminal 1 to prompt the user to capture an image. The user captures an image by using a camera of the first terminal 1. Hereinafter, the captured image including the diseased portion of the plant is also simply referred to as a “diseased portion image”. Note that an area and a capturing method of the diseased portion image are not particularly limited as long as the diseased portion is captured. For example, the diseased portion image may be a wide-area image obtained by imaging a farm field in which a crop having a diseased site recognized thereon is growing. When capturing the diseased portion image, the first terminal 1 acquires the first position information. The first terminal 1 transmits the first terminal specifying information, the first position information, and the diseased portion image to the diagnosis server 2 in association with each other. At the time of image capturing, the first terminal 1 is located in the vicinity of the plant, and thus the first position information can be regarded as information indicating a growth area of the plant.

When receiving the first terminal specifying information, the first position information, and the diseased portion image, the diagnosis server 2 diagnoses the type of disorder occurring on the plant based on the diseased portion image. As will be described in detail later, the diagnosis server 2 estimates the type of disorder based on the diseased portion image by using a trained model for disorder diagnosis. That is, the trained model outputs information indicating the type of disorder occurring on the plant as a diagnosis result. Hereinafter, the trained model for disorder diagnosis is referred to as a “diagnosis model”. The diagnosis server 2 transmits the diagnosis result to the first terminal 1. Finally, the first terminal 1 displays the diagnosis result. According to this, the first user can know the diagnosis result of the imaged plant. When the disorder diagnosis ends, the diagnosis server 2 transmits, to the prediction server 3, data in which the diagnosis result, the diagnosis date, and the first position information are collected (hereinafter, “referred to as diagnosis data”).

(Plant Disorder Prediction System 200)

When receiving the diagnosis data, the prediction server 3 accumulates the diagnosis data in a database (DB) as it is, or processes the diagnosis data partially and then accumulates it in a database (DB). Furthermore, the prediction server 3 constructs a trained model capable of predicting the possibility of occurrence of a disorder on the plant in an arbitrary area and on an arbitrary date by using the accumulated data. Hereinafter, the trained model is referred to as a “prediction model”. The construction of the prediction model is desirably completed before the prediction application is installed in the second terminal 4. The second terminal 4 transmits the second terminal specifying information and the second position information to the prediction server 3 according to the prediction application. By using the constructed prediction model, the prediction server 3 predicts the possibility of occurrence of the disorder on the plant in one or more areas including at least the area indicated by the second position information. The prediction server 3 transmits a part or all of the prediction results to the second terminal 4. The second terminal 4 displays the prediction result.

<<Main Part Configuration>>

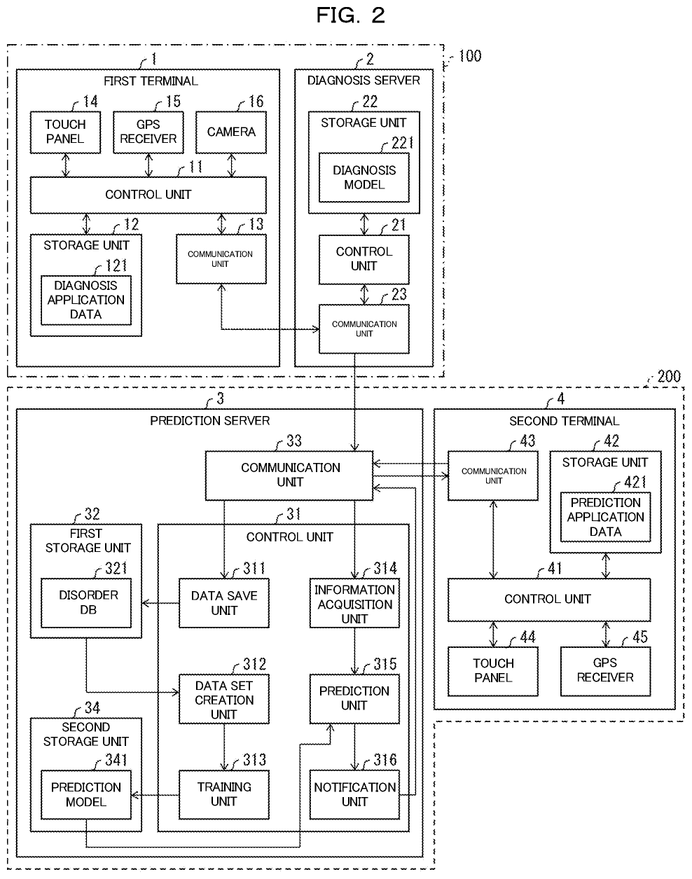

FIG. 2 is a block diagram illustrating a configuration of a main part of the plant disorder diagnosis system 100 and the plant disorder prediction system 200. As described above, the plant disorder diagnosis system 100 includes the first terminal 1 and the diagnosis server 2. Furthermore, the plant disorder prediction system 200 includes the prediction server 3 and the second terminal 4.

(First Terminal 1)

The first terminal 1 includes at least a control unit 11, a storage unit 12, a communication unit 13, a touch panel 14, a GPS receiver 15, and a camera 16. The communication unit 13 performs communication between the first terminal 1 and the diagnosis server 2. The touch panel 14 is a device in which a display device and an input device are integrated. The touch panel 14 receives a user's touch operation as an input operation. Furthermore, the touch panel 14 displays an image according to the control of the control unit 11. The camera 16 captures an image around the first terminal 1 according to the control of the control unit 11. The GPS receiver 15 receives a radio wave from a GPS (global positioning system) satellite. The GPS receiver 15 calculates a position of the first terminal 1 based on the received radio wave. The GPS receiver 15 outputs the first position information to the control unit 11. The reception and positioning of the radio wave in the GPS receiver 15 may be executed automatically or periodically, or may be executed in response to an instruction of the control unit 11.

The control unit 11 integrally controls the first terminal 1. The control unit 11 specifies a content of the user's input operation on the touch panel 14. Furthermore, the control unit 11 controls each unit of the first terminal 1 according to the specified content. For example, the control unit 11 starts the diagnosis application in response to the input operation. That is, the control unit 11 reads diagnosis application data 121 and executes processing. Note that the diagnosis application may be an application that requires user registration when the diagnosis application is used. In this case, for example, when the diagnosis application is started for the first time, the control unit 11 causes the first user to perform an operation for user registration by using the touch panel 14. The operation for the user registration is, for example, an operation of inputting a user name, a farming area, and the like. The control unit 11 stores the input information regarding the first user in the storage unit 12. Hereinafter, the information regarding the first user is referred to as “first user information”. Furthermore, the control unit 11 instructs the camera 16 to capture the diseased portion image according to the input operation of the user. Furthermore, the control unit 11 may instruct the GPS receiver 15 to perform positioning when transmitting the instruction to the camera 16. When acquiring the diseased portion image and the first position information, the control unit 11 transmits the diseased portion image to the diagnosis server 2 via the communication unit 13. At this time, the control unit 11 transmits the first terminal specifying information, the first position information, and the diseased portion image to the diagnosis server 2 in association with each other.

Note that in a case where the first user information is stored in the storage unit 12, the control unit 11 may use the first user information as the first terminal specifying information. That is, the control unit 11 may transmit the first user information, the first position information, and the diseased portion image to the diagnosis server 2 in association with each other. Furthermore, in a case where the first user information is transmitted to the diagnosis server 2, and the first user information includes information indicating a position or an area such as a farming area (hereinafter, referred to as area information), the first terminal 1 may transmit not the position information measured by the GPS receiver 15 but the area information included in the first user as the first position information. The control unit 11 also receives the diagnosis result from the diagnosis server 2. The control unit 11 displays the received diagnosis result on the touch panel 14 according to the diagnosis application.

The storage unit 12 is a storage device that stores various data necessary for the operation of the first terminal 1. The data stored in the storage unit 12 is added, updated, or deleted by the control unit 11. The storage unit 12 includes the diagnosis application data 121. The diagnosis application data 121 is program data of the diagnosis application. The storage unit 12 may also store the first user information.

(Diagnosis Server 2)

The diagnosis server 2 includes a control unit 21, a storage unit 22, and a communication unit 23. The communication unit 23 performs communication between the diagnosis server 2, and the first terminal 1 and the prediction server 3. The storage unit 22 is a storage device that stores various data necessary for the operation of the diagnosis server 2. The storage unit 22 stores a diagnosis model 221.

The diagnosis model 221 is a trained model obtained by training a correlation between the diseased portion image of the plant and the type of disorder in a machine learning manner. The diagnosis model 221 estimates the type of disorder indicated by the input diseased portion image and outputs the diagnosis result including information indicating the type. Note that a method of constructing the diagnosis model 221 is not particularly limited. Furthermore, the diagnosis model 221 may be a trained model capable of estimating presence or absence of occurrence of the disorder by using the diseased portion image. That is, the diagnosis model 221 may be a trained model that outputs the diagnosis result indicating that the type of disorder is “none” on some diseased portion image.

The control unit 21 integrally controls the diagnosis server 2. The control unit 21 receives the first terminal specifying information, the first position information, and the diseased portion image from the first terminal 1 via the communication unit 23. The control unit 21 inputs the diseased portion image to the diagnosis model 221 and acquires the type of disorder output from the diagnosis model 221 as a diagnosis result. The control unit 21 transmits the diagnosis result to the first terminal 1. The control unit 21 transmits the diagnosis result to the first terminal 1 indicated by the first terminal specifying information. According to this, even in a case where a plurality of the first terminals 1 are present in the plant disorder diagnosis system 100, the diagnosis result corresponding to the diseased portion image sent from each terminal can be returned to each of the first terminals 1. The control unit 21 also creates diagnosis data in which the first position information associated with the diseased portion image, the diagnosis result acquired using the diseased portion image, and the diagnosis date are collected. The control unit 21 transmits the diagnosis data to the prediction server 3. The diagnosis date may include not only a date but also at least one of year and month, or time. Furthermore, in a case where there is no particular description in the present specification, “day” and “date” indicating a period or a time point may include information of year, month, and time. Note that the diagnosis date may be acquired from a clocking unit (not illustrated) included in the diagnosis server 2 at the time of acquiring the diagnosis result. Alternatively, in a case where the data transmitted from the first terminal 1 to the diagnosis server 2 includes a date and time of capturing the diseased portion image, the date and time being measured by the first terminal 1, the diagnosis server 2 may specify the diagnosis date based on the date and time.

(Prediction Server 3)

The prediction server 3 includes a control unit 31, a first storage unit 32, a communication unit 33, and a second storage unit 34. The communication unit 33 performs communication between the prediction server 3, and the diagnosis server 2 and the second terminal 4. The first storage unit 32 is a storage device that stores various data necessary for the operation of the prediction server 3. For example, the first storage unit 32 stores a disorder DB 321. The disorder DB 321 is a DB including records in which information indicating an area to which the first position information belongs, a diagnosis date, and a diagnosis result are associated with each other in a case where the entire prediction target area is classified into predetermined areas. The records in the disorder DB 321 are added by a data save unit 311 to be described later. Furthermore, the disorder DB 321 is referred to and extracted by a data set creation unit 312. Note that in a case where the diagnosis data includes the first user information, each of the records in the disorder DB 321 may include at least a part of the first user information such as a user name.

The second storage unit 34 is a storage device that stores a prediction model 341. The prediction model 341 is data indicating an algorithm of the trained model. The prediction model 341 is constructed by a training unit 313 to be described later. Note that, in the present embodiment, the prediction model 341 has a structure of a neural network (NN: Neural Network). For example, the prediction model 341 may be a recurrent NN (RNN: Recurrent-type NN). However, the prediction model 341 may be a trained model constructed by applying another algorithm as long as it is a model capable of predicting the possibility of disorder occurrence. Note that, in a case of constructing a trained model of the NN as the prediction model 341, the prediction model 341 is preferably a multilayer NN that can be expected to have high specific accuracy. The second storage unit 34 may store an untrained model such as an untrained NN. Furthermore, the second storage unit 34 may store data indicating a weighting factor of the NN of the prediction model 341.

The control unit 31 integrally controls the prediction server 3. The control unit 31 includes the data save unit (data acquisition unit) 311, the data set creation unit 312, the training unit 313, an information acquisition unit (second position information acquisition unit) 314, a prediction unit 315, and a notification unit 316.

The data save unit 311 receives the diagnosis data from the diagnosis server 2 via the communication unit 33. The data save unit 311 stores one piece of the diagnosis data as one record in the disorder DB 321. Note that the data save unit 311 may process the diagnosis data according to a data format of the disorder DB 321 and then store the processed diagnosis data in the disorder DB 321. For example, the data save unit 311 may specify an area to which the first position information belongs based on the first position information, and store the area, the diagnosis date, and the diagnosis result, as one record, in the disorder DB 321.

The data set creation unit 312 extracts at least some of the records in the disorder DB 321, and creates training data used for machine learning of the prediction model 341 based on the extracted record. The data set creation unit 312 outputs the data set of the created training data to the training unit 313. Hereinafter, the data set of the training data is also simply referred to as a “data set”. Note that the data set creation timing is not particularly limited. For example, the data set creation unit 312 may create a data set when a predetermined number of records are accumulated in the disorder DB 321. Furthermore, in a case where the training unit 313 is configured to execute retraining of the prediction model 341, the data set creation unit 312 may create a data set when a predetermined number of new records having not been used for creating a data set so far are accumulated in the disorder DB 321.

The training unit 313 constructs the prediction model 341. The training unit 313 trains an untrained model in a machine learning manner by using the data set input from the data set creation unit 312. Note that the untrained model may be stored in the second storage unit 34 or may be held by the training unit 313. The method of machine learning may be appropriately determined according to the format of the prediction model 341 desired to be obtained, the number of data sets (that is, the number of records in the training data), and the content of each training data. For example, the training unit 313 reads an untrained model such as an NN algorithm and various weighting factors from the second storage unit 34. The training unit 313 causes the NN to execute supervised machine learning using each training data of the data set created by the data set creation unit 312. According to this, the training unit 313 can optimize the weighting factor or the like of the NN of the prediction model 341.

The information acquisition unit 314 acquires the second position information from the second terminal 4 and outputs the second position information to the prediction unit 315. The prediction unit 315 predicts the possibility of occurrence of the disorder based on information indicating an arbitrary date and an arbitrary area by using the prediction model 341. Specifically, when the prediction unit 315 inputs an arbitrary date and information indicating an arbitrary area to the prediction model 341, the prediction result is output from the prediction model 341. The prediction unit 315 outputs the prediction result to the notification unit 316.

The notification unit 316 notifies the second terminal 4 of the input prediction result via the communication unit 33. Note that the notification unit 316 may process at least a part of the prediction result input from the prediction unit 315 into an expression method and a data format for presenting to the second terminal 4. Note that the method of expressing the prediction result is not particularly limited. For example, the prediction unit 315 may specify one disorder that is most likely to occur and output the type of disorder as the prediction result. Furthermore, for example, the prediction unit 315 may output, as the prediction result, the type of disorder and an index value of an occurrence frequency of the disorder for a disorder having a possibility of occurrence higher than a predetermined threshold. Furthermore, the data format of the prediction result is not particularly limited. For example, the notification unit 316 may output the prediction result with text data or may output the prediction result with image data such as a circular graph. Furthermore, the expression method and the data format of the prediction result may be determined according to the specification of the second terminal 4.

(Second Terminal 4)

The second terminal 4 includes a control unit (second position information transmission unit, a prediction result reception unit, and display control unit) 41, a storage unit 42, a communication unit 43, a touch panel (display unit) 44, and a GPS receiver 45. The communication unit 43 performs communication between the first terminal 1 and the prediction server 3. The touch panel 44 has the same function as that of the touch panel 14, and receives a touch operation of the second user as an input operation. The GPS receiver 45 has the same function as that of the GPS receiver 15, and calculates the position of the second terminal 4. The GPS receiver 45 outputs the second position information to the control unit 41. The reception and positioning of the radio wave in the GPS receiver 45 may be executed automatically or periodically, or may be executed in response to an instruction from the control unit 41.

The control unit 41 integrally controls the second terminal 4. The control unit 41 specifies a content of the user's input operation on the touch panel 44. Furthermore, the control unit 41 controls each unit of the second terminal 4 according to the specified content. For example, the control unit 41 starts the prediction application in response to the input operation of the user. That is, the control unit 41 reads prediction application data 421 and executes processing. Note that the prediction application may be an application that requires user registration when the prediction application is used. In this case, for example, when the prediction application is started for the first time, the control unit 41 causes the second user to perform an operation for user registration by using the touch panel 44. The operation for the user registration is, for example, an operation of inputting a user name, a farming area, and the like. The control unit 41 stores the input information regarding the second user in the storage unit 42. Hereinafter, the information regarding the second user is referred to as “second user information”. Furthermore, when acquiring the second position information from the GPS receiver 45, the control unit 41 transmits the second position information to the prediction server 3. At this time, the control unit 41 transmits, to the prediction server 3, the second terminal specifying information that is information for specifying the second terminal 4 and the second position information in association with each other. The second terminal specifying information is, for example, an identification number unique to the second terminal 4.

Note that in a case where the second user information is stored in the storage unit 42, the control unit 41 may use the second user information as the second terminal specifying information. That is, the control unit 41 may transmit the second user information and the second position information to the prediction server 3 in association with each other. Furthermore, in a case where the second user information is transmitted to the prediction server 3 and the area information is included in the second user information, the second terminal 4 may not transmit the second position information. Furthermore, the control unit 41 receives the prediction result from the prediction server 3. The control unit 41 causes the touch panel 44 to display the received prediction result. Note that in a case where the data of the prediction result transmitted from the notification unit 316 includes voice data, the control unit 41 may cause a speaker (not illustrated) or the like of the second terminal 4 to output the voice.

The storage unit 42 is a storage device that stores various data necessary for the operation of the second terminal 4. The data stored in the storage unit 42 is added, updated, or deleted by the control unit 41. The storage unit 42 includes the prediction application data 421. The prediction application data 421 is program data of the prediction application. The storage unit 12 may also store the second user information.

Note that a function of the first terminal 1 and a function of the second terminal 4 may be realized by one terminal device as described above. In a case where the function of the first terminal 1 and the function of the second terminal 4 are realized by one terminal device, the control unit 11, the storage unit 12, the communication unit 13, the touch panel 14, and the GPS receiver 15 of the first terminal 1 also operate as the control unit 41, the storage unit 42, the communication unit 43, the touch panel 44, and the GPS receiver 45, respectively. Furthermore, the storage unit 12 includes the diagnosis application data 121 and the prediction application data 421. Furthermore, the storage unit 12 may store the first user information and the second user information.

<<Process Flow Related to Construction of Prediction Model>>

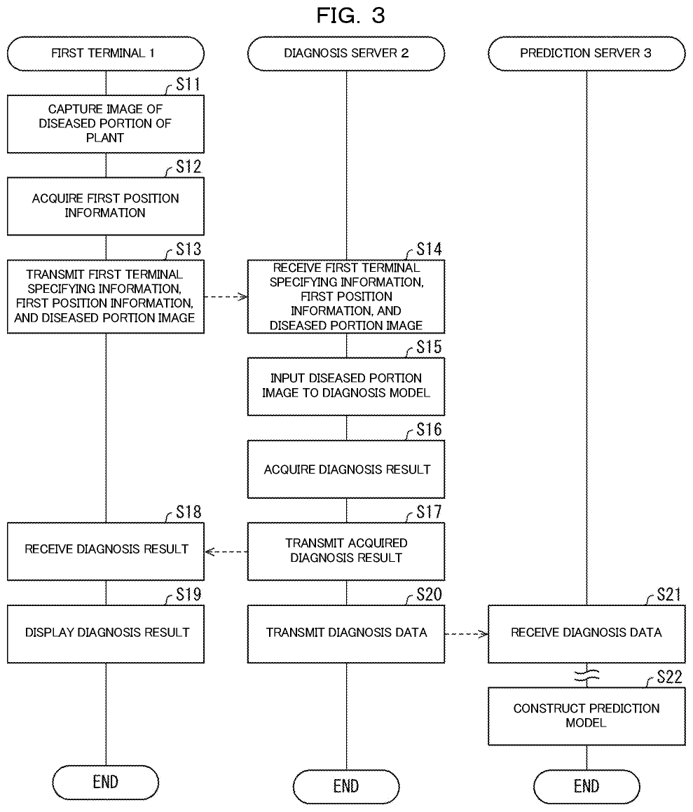

FIG. 3 is a sequence diagram illustrating a flow of a model construction process in the plant disorder prediction system 200. Here, the “model construction process” means a series of processes for constructing the prediction model 341. As described above, in the model construction process, the prediction model 341 is constructed using various data obtained at the time of diagnosis of the disorder in the plant disorder diagnosis system 100. Therefore, in FIG. 3, S11 to S18, which are processes related to the plant disorder diagnosis system 100, are also described and explained.

A first user who has found in the plant a mutation predicted to be caused by the disorder attempts to obtain the diagnosis result of the disorder by using the diagnosis application. Specifically, the first user starts the diagnosis application of the first terminal 1, and performs an input operation for capturing an image of the diseased portion of the plant on the first terminal 1 according to an instruction of the diagnosis application. The control unit 11 of the first terminal 1 operates the camera 16 in response to the input operation. The camera 16 captures an image including the diseased portion of the plant (S11). The camera 16 outputs the captured image, that is, the diseased portion image to the control unit 11. Furthermore, the GPS receiver 15 acquires the first position information by receiving a signal from a GPS satellite (S12). The GPS receiver 15 outputs the acquired first position information to the control unit 11. Note that S12 may be executed before S11 or in parallel to S11. Furthermore, in a case where information such as a farming area of the first user information is used as the first position information, the control unit 11 may read the first user information from the storage unit 12 instead of processing of S12. The control unit 11 transmits the first terminal specifying information, the first position information, and the diseased portion image to the diagnosis server 2 in association with each other (S13).

The communication unit 23 of the diagnosis server 2 receives the first terminal specifying information, the first position information, and the diseased portion image (S14). The communication unit 23 outputs the received data to the control unit 21. The control unit 21 diagnoses a disorder of the plant based on the diseased portion image by using the received data and the diagnosis model 221. Specifically, the control unit 21 inputs the diseased portion image to the diagnosis model 221 (S15) and acquires the diagnosis result output from the diagnosis model 221 (S16). The control unit 21 transmits the acquired diagnosis result to the first terminal 1 via the communication unit 23 (S17). When receiving the diagnosis result (S18), the control unit 11 of the first terminal 1 displays the diagnosis result on the touch panel 14 according to the provisions of the diagnosis application data 121 (S19). Furthermore, after the processing of S16, the diagnosis server 2 transmits the diagnosis data to the prediction server 3 (S20). Note that the processing timing of S20 is not particularly limited as long as it is after S16. For example, S20 may be executed before S17.

The data save unit 311 of the prediction server 3 receives the diagnosis data via the communication unit 33 (S21). The data save unit 311 stores one piece of the diagnosis data as it is or after processed, as one record, in the disorder DB 321. As described above, every time the first user performs the disorder diagnosis for the plant by using the diagnosis application, the diagnosis data is generated and the number of records in the disorder DB 321 increases. Thereafter, at a predetermined timing, the data set creation unit 312 reads at least some records in the disorder DB 321 and creates a data set of the training data for machine learning. The data set creation unit 312 outputs the data set to the training unit 313. The training unit 313 trains the prediction model 341 stored in the second storage unit 34 in a machine learning manner by using the data set (S22). According to this, the prediction model 341 trained in the machine learning manner is constructed.

According to the above-described processing, it is possible to construct a prediction model that predicts the possibility of occurrence of the disorder in an arbitrary area and on an arbitrary date. Accordingly, it is possible to construct the prediction model capable of accurately predicting the possibility of occurrence of the disorder of the plant. Furthermore, according to the above-described processing, the data set used for constructing the prediction model 341 is generated from the disorder DB 321. The record in the disorder DB 321, that is, the diagnosis data increases every time the first user performs the disorder diagnosis by using the diagnosis application of the first terminal 1. Therefore, according to the above-described process, it is possible to collect a large number of pieces of diagnosis data necessary for constructing the prediction model 341. Furthermore, while the diagnosis application is used, new diagnosis data can be always obtained.

Note that the processes related to the plant disorder diagnosis system 100 and the processes related to the plant disorder prediction system 200 may be executed discontinuously. That is, the processes of S11 to S19 and the processes of S20 to S22 may be performed at different timings. Furthermore, the processes up to S21, the creation of the data set, and the processing of S22 may be performed at different timings. Furthermore, the diagnosis server 2 may transmit the diagnosis data to the prediction server 3 every time new diagnosis data is obtained, or may collectively transmit a plurality of pieces of the diagnosis data to the prediction server 3 after acquiring a plurality of pieces of diagnosis data. For example, the diagnosis server 2 may repeat the processes of S11 to S19 a plurality of times, and then in S20, collectively transmit the diagnosis data obtained by repeating the processes a plurality of times. Furthermore, in a case where the first user information includes the area information and the first terminal 1 transmits the first user information to the diagnosis server 2, the diagnosis server 2 may include the first user information in the diagnosis data. In this case, the prediction server 3 may store a record in which the diagnosis result, the diagnosis date, and the area information are associated with each other in the disorder DB 321.

<<Process Flow Related to Disorder Prediction>>

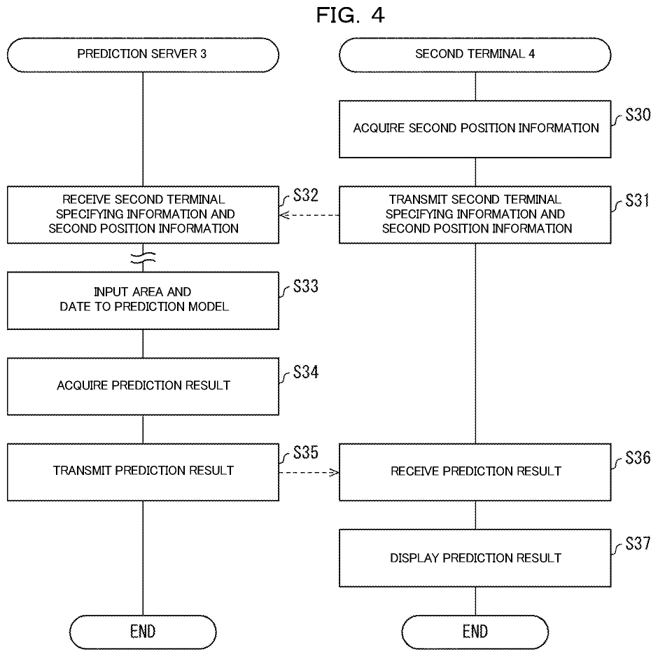

FIG. 4 is a sequence diagram illustrating a flow of a prediction process in the plant disorder prediction system 200. Here, the “prediction process” means a series of processes for predicting the possibility of disorder occurrence at a certain position or area by using the prediction model 341. Note that, in FIG. 4, as an example, the prediction process is executed in a case where the user starts the prediction application of the second terminal 4 and instructs prediction of disorder occurrence of the plant by using the touch panel 44. The control unit 41 of the second terminal 4 acquires the second position information from the GPS receiver 45 (S30), and transmits the second position information to the prediction server 3 (S31). The information acquisition unit 314 of the prediction server 3 receives the second position information via the communication unit 33 (S32). The information acquisition unit 314 outputs the second position information to the prediction unit 315. The prediction unit 315 specifies an area indicated by the second position information, and inputs information indicating the area and a predetermined date to the prediction model 341 (S33). According to this, the prediction result for the possibility that various disorders occur in the area indicated by the second information and on a predetermined date is output. Note that in a case where the area information is included in the second user information and the second terminal 4 transmits the second user information to the prediction server 3, the prediction server 3 may obtain the prediction result by inputting the area information and the predetermined date to the prediction model 341. The prediction unit 315 acquires the prediction result (S34), and outputs the prediction result to the notification unit 316. The notification unit 316 transmits the prediction result to the second terminal 4 (S35). The control unit 41 of the second terminal 4 receives the prediction result via the communication unit 43 (S36). The control unit 41 causes the touch panel 44 to display the prediction result (S37).

According to the above-described process, it is possible to predict the possibility of occurrence of the disorder in an arbitrary area and on an arbitrary date by using the prediction model 341. As described above, the prediction model 341 is constructed based on a sufficient number of pieces of the diagnosis data collected from time to time. Therefore, according to the above-described process, it is possible to accurately predict the possibility of occurrence of the disorder on the plant. Furthermore, according to the above-described process, the possibility of occurrence of the disorder on the plant is predicted using the prediction model 341. Therefore, even in a case where the diagnosis data for the entire target area for prediction cannot be prepared as in a case where the possibility of occurrence of the disorder is predicted on a rule-based basis using the past diagnosis data itself, the possibility of occurrence of the disorder can be predicted.

Note that the second terminal 4 according to the present embodiment may measure the second position information periodically by using the GPS receiver 45. The control unit 41 may periodically transmit the second terminal specifying information and the second position information to the prediction server 3. In this case, the information acquisition unit 314 of the prediction server 3 periodically performs reception (acquisition) process in S32. Furthermore, the prediction unit 315 executes the processes of S33 to S34 as described above every time the second position information is acquired. The notification unit 316 determines whether or not the acquired prediction result, that is, the possibility of occurrence of the disorder satisfies a predetermined condition as described above. In a case where a predetermined condition is satisfied, the notification unit 316 transmits the prediction result to the second terminal 4. On the other hand, in a case where the predetermined condition is not satisfied, the notification unit 316 does not transmit the prediction result and ends the process. That is, the prediction server 3 does not execute the process of S35, and thus the second terminal 4 does not execute the processes of S36 and S37.

Note that the processing of S33, that is, a prediction timing in the prediction unit 315 is not particularly limited. For example, in S33, the prediction unit 315 may predict the possibility of occurrence of the disorder on a predetermined date (for example, the next day) for the entire target area for prediction once a day. The prediction unit 315 may output the prediction result for the area corresponding to the second position information among the prediction results and the second terminal specifying information to the notification unit 316. The notification unit 316 may transmit the prediction result input from the prediction unit 315 to the second terminal 4 indicated by the second terminal specifying information. Alternatively, the prediction unit 315 may transmit the prediction result for the entire area to notification unit 316. In this case, the notification unit 316 may acquire the second position information and the second terminal specifying information from the information acquisition unit 314, and transmit the prediction result for the area indicated by the second position information to the second terminal 4 indicated by the second terminal specifying information. Furthermore, in a case where the possibility of occurrence of the disorder in the area indicated by the second position information satisfies a predetermined condition, the notification unit 316 may transmit the prediction result to the second terminal 4 indicated by the second terminal specifying information. For example, in a case where a value indicating the possibility of occurrence of the disorder is equal to or greater than a predetermined threshold (for example, a disorder occurrence probability is 50% or greater), the notification unit 316 may determine that the predetermined condition is satisfied. Furthermore, the notification unit 316 may determine that the “predetermined condition” is satisfied in a case where a predetermined period has elapsed after the prediction result is transmitted to the second terminal 4 indicated by the previous second terminal specifying information.

In this manner, the prediction server 3 can execute the prediction process without an instruction of the second user by periodically acquiring the second position information and performing the prediction process or by periodically predicting the entire target area for prediction. Furthermore, the prediction server 3 performs notification in a case where the prediction result satisfies a predetermined condition. According to this, unnecessary notification to the second terminal 4 can be omitted. Furthermore, it is possible to notify the second user of the prediction result at a necessary timing.

### MODIFICATION EXAMPLE

The diagnosis server 2 may store a disorder countermeasure information DB in the storage unit 22. In the disorder countermeasure information DB, a type of disorder and a countermeasure for preventing or resolving the disorder are recorded in association with each other. In a case where the disorder is disease and insect pest, the “countermeasure” is, for example, a type of drug effective for the disorder. Furthermore, in a case where the disorder is a physiological disorder, the “countermeasure” is mulching, installation of a sunshade, a type of fertilizer effective for resolving the physiological disorder. Note that the disorder countermeasure information DB may be shared between the diagnosis server 2 and the prediction server 3. For example, the disorder countermeasure information DB of the diagnosis server 2 may also be accessible from the prediction server 3. Furthermore, the disorder countermeasure information DB may be transmitted from the diagnosis server 2 to the prediction server 3 and stored in the first storage unit 32 of the prediction server 3. The prediction server 3 may acquire the disorder countermeasure information DB from the diagnosis server 2 periodically or at a specific timing, and update the disorder countermeasure information DB held by the prediction server 3.

Furthermore, the first user information and the second user information may be appropriately updated after the user registration. For example, the control unit 41 may cause the touch panel 44 to display a predetermined input screen after the prediction application is started. The second user may add, update, or delete various pieces of information included in the second user information by using the touch panel 44. For example, the control unit 41 may cause the second user to input the type of the disorder countermeasure taken by the second user in order for the second user to prevent or resolved the disorder and the date of taking the countermeasure. These pieces of information may be associated with each other to form disorder countermeasure history data. The disorder countermeasure history data is included in the second user information and stored. Also for the diagnosis application of the first terminal 1, the first user information may be added, updated, or deleted in the same manner as for the prediction application of the second terminal 4.

(Prediction Result Display Screen)

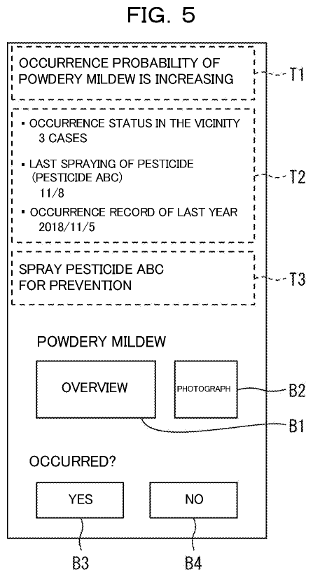

Various pieces of information may be displayed on the touch panel 44 of the second terminal 4 together with the prediction result. FIG. 5 is a diagram illustrating an example of the display screen showing the prediction result, the display screen being displayed on the display surface of the touch panel 44 by executing the process of S37 in FIG. 4. Hereinafter, the display screen showing the prediction result is referred to as a “prediction result display screen”. In the example of FIG. 5, the prediction result display screen includes a text T1 indicating a prediction result, a text T2 indicating various pieces of information related to the prediction result, and a text T3 indicating a disorder countermeasure method in response to the prediction result. The content of the text T2 may be appropriately determined according to the information held by the second terminal 4. For example, in a case where the second user information includes the disorder countermeasure history, information related to the disorder countermeasure history such as a type of pesticide sprayed last time and date and time of spraying may be displayed as the text T2. Furthermore, the notification unit 316 may transmit various pieces of information to the second terminal 4 together with the prediction result. For example, the notification unit 316 may specify, from the disorder DB 321, whether or not there is a record indicating the type of certain disorder included in the prediction result in a past first period. Here, the certain disorder is, for example, a disorder predicted to be most likely to occur in the prediction result. The first period as an example includes 15 days before and 15 days after the day one year before the prediction target date. In a case where there is a record indicating the certain disorder, the notification unit 316 may transmit, to the second terminal 4, the diagnosis date (that is, the occurrence date of the disorder) in the record. In this case, the control unit 41 of the second terminal 4 may cause the touch panel 44 to display an occurrence record of the certain disorder last year as the text T2 as illustrated in FIG. 5.

Furthermore, for example, the notification unit 316 may extract a record of the area indicated by the second position information in a second period from the disorder DB 321 and calculate the times of occurrence of the certain disorder in the second period. The second period is, for example, from the date set as the prediction target to the date 10 days before the prediction target date. The times of occurrence may be transmitted to the second terminal 4. Note that in a case where the prediction result indicates a plurality of types of disorder, the notification unit 316 may calculate the times of occurrences of each disorder. The notification unit 316 may transmit information indicating the calculated times of occurrence of each disorder to the second terminal 4. In this case, the control unit 41 of the second terminal 4 may cause the touch panel 44 to display an occurrence status of the certain disorder in the vicinity as the text T2 as illustrated in FIG. 5. Furthermore, the notification unit 316 may specify a countermeasure to prevent or resolve the certain disorder by referring to the disorder countermeasure information DB which is stored in the first storage unit 32 or shared with the diagnosis server 2. The notification unit 316 may transmit information indicating the countermeasure to the second terminal 4. In this case, the control unit 41 of the second terminal 4 may cause the touch panel 44 to display the countermeasure corresponding to the type of disorder as the text T3 as illustrated in FIG. 5.

Furthermore, as illustrated in FIG. 5, the prediction result display screen may include a button B1 for searching for an overview of a disorder specified as a prediction result in a network, a button B2 for searching for an image of a plant on which the disorder has occurred and displaying the image, and feedback buttons B3 and B4. The feedback buttons B3 and B4 are buttons for feeding back an actual status of disorder occurrence to the prediction server 3 in comparison with the prediction result. When the feedback button B3 displayed on the touch panel 44 is pressed, the control unit 41 generates feedback information and transmits the feedback information to the prediction server 3. More specifically, the control unit 41 acquires the date when the feedback button B3 is pressed and the second position information. The control unit 41 generates feedback information including the acquired date and second position information, and information indicating the type of disorder that has occurred. Also in a case where the feedback button B4 displayed on the touch panel 44 is also pressed, the feedback information is generated in a similar manner. However, in a case where the button B4 is pressed, the type of disorder that has occurred is “none”. The control unit 41 transmits the generated feedback information to the prediction server 3 via the communication unit 43. The information acquisition unit 314 of the prediction server 3 acquires the feedback information. The information acquisition unit 314 stores the feedback information in the first storage unit 32. At this time, the feedback information may be stored as one record of the disorder DB 321, or may be separately stored as a DB of the feedback information. According to this, the feedback information from the second terminal 4 is accumulated in the first storage unit 32 each time.

(Retraining)

The prediction server 3 may retrain the prediction model 341. For example, the data set creation unit 312 of the prediction server 3 may create a new data set based on the disorder DB 321 every time a predetermined period elapses, for example, once a month, and output the data set to the training unit 313. The training unit 313 may retrain the prediction model 341 by using the newly created data set. Note that in a case where the prediction server 3 acquires and accumulates the feedback information as described above, the training unit 313 may retrain the prediction model 341 by using the accumulated feedback information. Furthermore, at the time of retraining, a data set for retraining may be created using both the feedback information and the disorder DB 321. As described above, new data can be applied to the prediction algorithm of the prediction model 341 by retraining the prediction model 341. Therefore, accuracy of the prediction using the prediction model 341 can be improved. Note that the specific method of retraining is not particularly limited. Note that the data set creation unit 312 may not extract a record used for previous training when newly creating a data set. That is, only the newly increased record may be used as the training data.

(Mapping of Prediction Result)

The first storage unit 32 of the prediction server 3 may store map data of the entire target area for prediction as a map DB. A method of acquiring the map DB in the prediction server 3 is not particularly limited. For example, the prediction server 3 may appropriately download the latest map DB via the Internet. The notification unit 316 may map the prediction result for each area acquired from the prediction unit 315 (for example, the occurrence probability of the disorder) into a map image indicated by the map DB and deliver the map image as the prediction result to the second terminal 4. For example, the notification unit 316 may draw lines on a map image of the entire target area for prediction by dividing the area in the prediction, and color-code each divided area according to the occurrence probability of a specific disorder. The color-coded map image may be transmitted to the second terminal 4. According to this, the second terminal 4 can display the map image in which the occurrence probability of the specific disorder in each area can be seen at a glance. Therefore, the second user can grasp the distribution of the occurrence probability of the disorder at a glance.

### Second Embodiment

Another embodiment of the present invention will be described below. Note that for convenience of description, members having the same functions as the members described in the above-described embodiment are denoted by the same reference numerals, and the description thereof will not be repeated. The same applies to the following embodiments.

<<Main Part Configuration>>

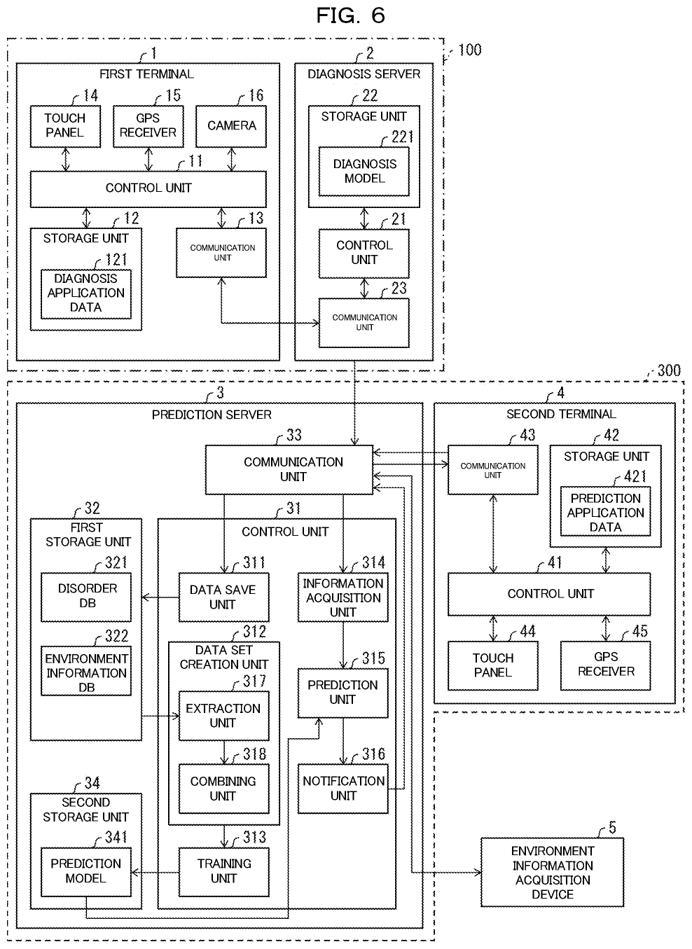

FIG. 6 is a block diagram illustrating a configuration of a main part of various systems (plant disorder diagnosis system 100 and plant disorder prediction system 300) according to the present embodiment. Since the plant disorder diagnosis system 100 according to the present embodiment has the same configuration and processing contents as those of the plant disorder diagnosis system 100 according to the first embodiment, the description thereof will not be repeated. The plant disorder prediction system 300 is different from the plant disorder prediction system 200 according to the first embodiment in that one or more environment information acquisition devices 5 are provided.

The environment information acquisition device 5 is a generic term for devices that collect environment information and provide the environment information to a prediction server 3. Here, the “environment information” indicates various types of information regarding a growth environment of the plant. For example, the environment information indicates, for example, weather information and information regarding soil. More specifically, the “weather information” indicates, for example, weather, a solar radiation amount per unit time, a solar radiation intensity, precipitation per unit time, a wind direction, a wind speed, a temperature (for example, a daily minimum temperature, a daily maximum temperature, a daily average temperature, and the like), humidity, an accumulated temperature, and the like. Furthermore, the “information regarding soil” is information indicating a soil temperature, a soil moisture amount, and a soil pH value in each area. A specific mode of the environment information acquisition device 5 is not particularly limited. For example, the environment information acquisition device 5 may be a server related to the operation of a site that provides various weather information such as a weather forecast service. Furthermore, for example, the environment information acquisition device 5 may be a log server or a terminal device that collects and manages environment information obtained from a measurement terminal such as various sensors installed in a greenhouse, or the measurement terminal itself. The environment information acquisition device 5 transmits the environment information to the prediction server 3 periodically or in response to a request from the prediction server 3. Note that there may be a plurality of environment information acquisition devices 5. For example, a certain environment information acquisition device 5 may transmit information indicating the weather and the precipitation to the prediction server 3, and another environment information acquisition device 5 may transmit information indicating the accumulated temperature and the soil temperature to the prediction server 3.

The prediction server 3 according to the present embodiment is different from the prediction server 3 according to the first embodiment in that a first storage unit 32 includes an environment information DB 322 and a data set creation unit 312 includes an extraction unit 317 and a combining unit 318. A data save unit 311 of the prediction server 3 according to the present embodiment acquires environment information (first environment information) from the environment information acquisition device 5 via a communication unit 33. The data save unit 311 stores the acquired information in the environment information DB 322. The environment information DB 322 is data in which a date, an area or a position, and environment information on the date and in the area or the position are associated with each other. The type of environment information may be changed according to the type of the environment information acquired by the prediction server 3 from the environment information acquisition device 5.

<<Model Construction Process>>

The data set creation unit 312 according to the present embodiment creates a data set based on a disorder DB 321 and the environment information DB 322. The extraction unit 317 of the data set creation unit 312 reads at least a part of the record in the disorder DB 321 as a record group used for creating a data set. Hereinafter, the record group is referred to as a “use target record group”. The extraction unit 317 further extracts a record corresponding to the date and position indicated by each record of the use target record group from the environment information DB 322. Hereinafter, the record group extracted from the environment information DB 322 by the extraction unit 317 is referred to as a “corresponding record group”. Note that in a case where a data format of the first position information of the disorder DB 321 is different from a data format of the information indicating the area or the place in the environment information DB 322, the extraction unit 317 specifies a corresponding record group while specifying which of the information indicating the area or the place in the environment information DB 322 the first position information of each record corresponds to.

The extraction unit 317 outputs the use target record group and the corresponding record group to the combining unit 318. The combining unit 318 combines each record of the use target record group with the record of the corresponding record group corresponding to the record of the use target record group. According to this, a plurality of records in which a date and an area are associated with the diagnosis result (that is, the type of disorder), and environment information on the date and in the area are generated. The data set creation unit 312 outputs a plurality of the generated records as a data set to the training unit 313. The training unit 313 trains a prediction model 341 in a machine learning manner by using the input data set. According to this, it is possible to train the prediction model 341 about a correlation between the first position information, diagnosis date, and environment information and the diagnosis result in a machine learning manner. According to this, it is possible to construct the prediction model 341 that predicts the possibility of occurrence of the disorder on an arbitrary date, in an arbitrary place and under an arbitrary environment condition. Therefore, it is possible to predict the possibility of occurrence of the disorder more accurately on the plant.

Note that the environment information acquisition device 5 may supply the environment information (first environment information) to the first terminal 1. For example, the control unit 11 of the first terminal 1 may acquire the first environment information from the environment information acquisition device 5 when using the diagnosis application. The control unit 11 may transmit the first terminal specifying information, the first position information, the diseased portion image, and the first environment information to the diagnosis server 2 in association with each other. In this case, the diagnosis server 2 transmits the diagnosis data including the first environment information to the prediction server 3. The data save unit 311 of the prediction server 3 acquires the first environment information included in the diagnosis data. The subsequent processes are as described above. In this case, the prediction server 3 may not directly receive the environment information from the environment information acquisition device 5.

<<Prediction Process>>

The information acquisition unit 314 according to the present embodiment acquires environment information (second environment information) from the environment information acquisition device 5. The information acquisition unit 314 may acquire the second environment information for each area for which the possibility of occurrence of the disorder can be predicted. The information acquisition unit 314 outputs the second position information and the second environment information to the prediction unit 315. Acquisition timings for these information may be independent. By inputting a predetermined date, a predetermined area, and environment information to the prediction model 341, the prediction unit 315 predicts the possibility of occurrence of the disorder on the predetermined date, in the predetermined area, and under an environment condition indicated by the received environment information. According to this, it possible to predict the possibility of occurrence of the disorder more accurately in consideration of the environment condition. Note that the predetermined date may be a current date or a future date. Furthermore, the predetermined area may be an area indicated by the second position information. In a case where the future date is input, the prediction unit 315 may predict, by using the prediction model 341, the possibility of occurrence of the disorder on the plant under the assumption that an environment condition at a certain future day and in the predetermined area would be the environment condition indicated by the received environment information. Furthermore, the prediction unit 315 may be capable of acquiring future environment information such as a weekly weather forecast from the environment information acquisition device 5. In this case, when the prediction unit 315 determines a predetermined date (future date), the information acquisition unit 314 acquires the environment information on the date from the environment information acquisition device 5. The prediction unit 315 predicts the possibility of occurrence of the disorder on the plant by inputting a certain future date, the second position information, and the environment information on the future date to the prediction model 341. According to this, it possible to predict the possibility of occurrence of the disorder under a specific environment condition on a future date and in a predetermined area.

Note that the environment information acquisition device 5 may supply the environment information (second environment information) to a second terminal 4. For example, the control unit 41 of the second terminal 4 may acquire the environment information from the environment information acquisition device 5 when the second terminal specifying information and the second position information are transmitted. The control unit 41 may transmit the second terminal specifying information, the second position information, and the environment information to the prediction server 3 in association with each other. In this case, the information acquisition unit 314 of the prediction server 3 acquires the second terminal specifying information, the second position information, and the environment information from the second terminal 4. The subsequent processes are as described above.

### Third Embodiment

The prediction unit 315 of the prediction server 3 according to each of the above-described embodiments may correct the prediction result output from the prediction model 341. For example, the prediction unit 315 may correct the prediction result of the prediction model 341 according to the times of occurrence of each disorder during a predetermined period in a predetermined area, the times of occurrence being calculated from the record of the disorder DB 321. In this case, the control unit 31 extracts a record whose diagnosis date is within a predetermined period from the disorder DB 321 at a predetermined timing. By counting the diagnosis result (that is, the type of disorder) indicated by the extracted record, the times of occurrence of each disorder within the predetermined period is calculated. The control unit 31 may cause the first storage unit 32 to store the calculated times of occurrences of each disorder. Note that a method of setting a predetermined period is not particularly limited. For example, the “predetermined period” may be a predetermined period retroactive from the present, such as one month from the day on which the prediction is executed. Furthermore, for example, the “predetermined period” may be a month and a day one year ago which is the same as the month and the day when the prediction is executed. The prediction unit 315 of the prediction server 3 corrects the prediction result of the prediction model 341 after the prediction using the prediction model 341. The correction method is not particularly limited. For example, the prediction unit 315 may increase the possibility of the disorder occurrence for the disorder that occurs a large number of times within a predetermined period. The prediction unit 315 outputs the corrected prediction result to the notification unit 316, and the notification unit 316 notifies the second terminal 4 of the corrected prediction result.

### Fourth Embodiment

The plant disorder prediction system according to the present invention may calculate an effect value of the disorder countermeasure taken by the second user on a target day for prediction. A value indicating the possibility of occurrence of the disorder, the value being predicted by the prediction process, may be corrected according to the effect value. Furthermore, the plant disorder prediction system according to the present invention may have a model formula for calculating the effect value for each disorder countermeasure. Furthermore, in the model formula, a factor or the like may be appropriately tuned by machine learning using information that indicates a disorder countermeasure history of the second user and an occurrence record of the disorder.

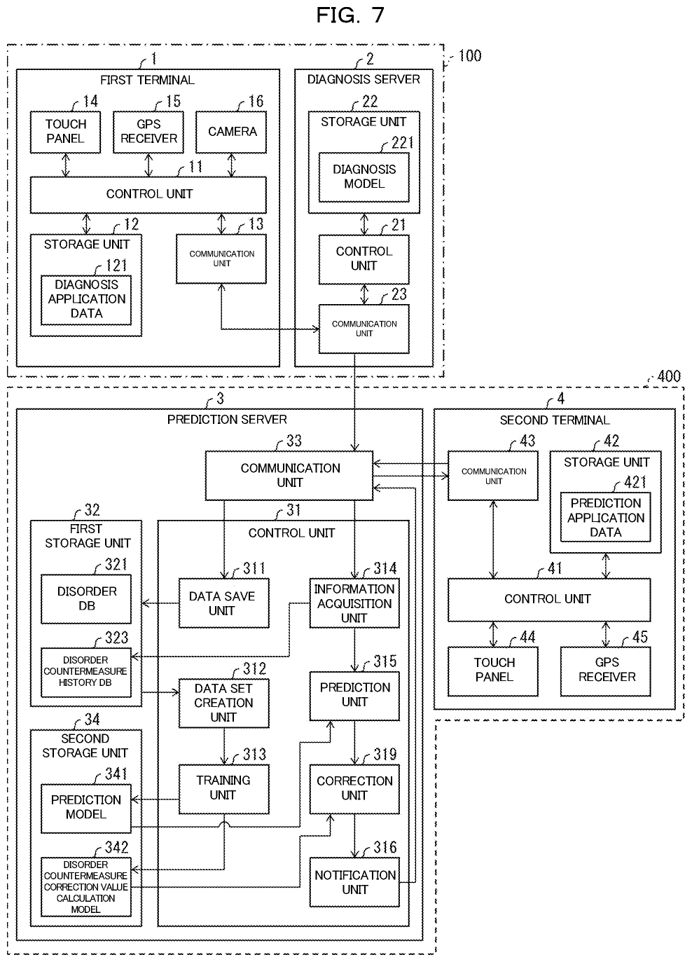

FIG. 7 is a block diagram illustrating a configuration of a main part of various systems (plant disorder diagnosis system 100 and plant disorder prediction system 400) according to the present embodiment. A first storage unit 32 of a prediction server 3 according to the present embodiment stores a disorder countermeasure history DB 323. Furthermore, a control unit 31 includes a correction unit 319. Furthermore, a second storage unit 34 includes a disorder countermeasure correction value calculation model 342. Furthermore, a storage unit 42 of a second terminal 4 according to the present embodiment stores second user information, and the second user information includes disorder countermeasure history data. Furthermore, the second terminal 4 transmits the second user information to the prediction server 3 together with or instead of second position information. An information acquisition unit 314 of the prediction server 3 stores the disorder countermeasure history data included in the second user information in the disorder countermeasure history DB 323.

The disorder countermeasure history DB 323 is a DB that stores types of disorder countermeasure and a date when the countermeasure is taken in association with each other. The DB may collectively store the disorder countermeasure history data acquired from a plurality of the second terminals 4. The disorder countermeasure correction value calculation model 342 is a model formula for each type of disorder and each type of disorder countermeasure, and is a model formula for calculating a value of a countermeasure effect (an effect value) on and after the day on which the disorder countermeasure is taken. In the present embodiment, the higher the effect value, the higher the effect of the disorder countermeasure (the effect continues).

A prediction unit 315 outputs a prediction result and the second user information to a correction unit 319. The correction unit 319 corrects the prediction result indicated by the second user information according to an execution date of the disorder countermeasure and the type of countermeasure, which are indicated by the disorder countermeasure history indicated by the second user information. The correction unit 319 reads the disorder countermeasure correction value calculation model 342 corresponding to the type of disorder and the type of disorder countermeasure, and calculates an effect value by inputting the date when the disorder countermeasure has been taken to the model formula. The correction unit 319 corrects the prediction result by using the effect value. For example, the correction unit 319 obtains the corrected prediction result by subtracting the calculated effect value from a value indicating a possibility of occurrence of the disorder indicated by the prediction result (occurrence probability). The correction unit 319 outputs the corrected prediction result to a notification unit 316. According to this, the possibility of occurrence of the disorder can be predicted in consideration of the effect of the disorder countermeasure taken by the second user. That is, more accurate prediction becomes possible.

Furthermore, the first storage unit 32 of the prediction server 3 may store the feedback information DB described in the first embodiment. A data set creation unit 312 according to the present embodiment may create a data set in which a record of the disorder countermeasure history DB 323 is associated with a record of the feedback information DB, that is, an actual disorder occurrence result. A training unit 313 may retrain each disorder countermeasure correction value calculation model 342 by using the data set. As long as a value of a factor of the disorder countermeasure correction value calculation model 342 can be tuned, a format of the data set and a method of retraining are not particularly limited. As described above, by tuning the model formula for calculating the effect value based on a history of the disorder countermeasure (that is, an execution record of the countermeasure) and a disorder occurrence result, the calculation accuracy of the effect value can be further improved.

### MODIFICATION EXAMPLE

The prediction server 3 according to each of the above-described embodiments may be divided into a DB server that stores various DBs and a processing server that executes model construction process and prediction process. In a case where the DB server and the processing server are separated, these servers are connected to each other in a wired or wireless manner, and data is transmitted and received. Furthermore, the DB server includes at least the first storage unit 32 illustrated in FIG. 2. Furthermore, the processing server includes at least the control unit 31, a communication unit 33, and the second storage unit 34. Furthermore, the processing server may be divided into a prediction model construction server that executes the model construction process and a prediction model use server that stores a prediction model 341 constructed by the construction server and executes prediction process. In this case, the prediction model construction server includes at least the communication unit 33, the control unit 31 including the data save unit 311, the data set creation unit 312 and the training unit 313, and the second storage unit 34. Furthermore, the prediction model use server includes at least the communication unit 33, the control unit 31 including the information acquisition unit 314, the prediction unit 315 and the notification unit 316, and the first storage unit 32 that stores the trained prediction model 341.

Furthermore, the first terminal 1 according to each of the above-described embodiments may transmit a name of the imaged plant to a diagnosis server 2, together with first terminal specifying information, first position information, and a diseased portion image. Note that a control unit 11 acquires the name of the plant by causing a user to input the name of the plant through the touch panel 14. The diagnosis server 2 may transmit, to the prediction server 3, diagnosis data including the name of the imaged plant, that is, the plant to be diagnosed. In this case, the name of the plant is also stored as a parameter of each record in a disorder DB 321. Therefore, a parameter of a data set created by the data set creation unit 312 also includes the name of the plant. The training unit 313 trains the prediction model 341 about the data set in a machine learning manner. According to this, it is possible to train the prediction model 341 about a correlation between the first position information, diagnosis date and the name of the plant to be diagnosed, and the diagnosis result. On the other hand, in the prediction processing, the information acquisition unit 314 of the prediction server 3 may acquire the name of the target plant for prediction, as the second user information, from the second terminal 4. The information acquisition unit 314 transmits the acquired various types of information to the prediction unit 315. The prediction unit 315 causes the prediction model 341 to predict the possibility of occurrence of the disorder by inputting a predetermined date, the second position information, the name of the target plant for prediction to the prediction model 341. In this manner, accuracy of the prediction result can be improved by constructing the prediction model 341 in consideration of the name of the plant and executing the prediction processing using the prediction model 341.

Furthermore, in a case where the data save unit 311 acquires the first user information including the user name, and in a case where the information acquisition unit 314 acquires the second user information including the user name from the second terminal 4, the prediction unit 315 may set the corrected prediction result, which is corrected from the prediction result of the prediction model 341, as a prediction result to be transmitted to the second terminal 4 according to the result obtained by searching the disorder DB 321 with the user name indicated by the second user information. For example, when searching the disorder DB 321 with the user name indicated by the second user information among the prediction results, the prediction unit 315 may increase the possibility of occurrence of the disorder indicated by the diagnosis result having the largest hit count. According to this, it is possible to predict a higher occurrence possibility for the disorder that is likely to be caused to occur by the second user. Therefore, accuracy of the prediction result to be transmitted to the second terminal 4 can be improved.

In each of the above-described embodiments, the diagnosis server 2 may receive, from the first terminal 1, place information indicating whether a place where a diseased portion image is captured is an open field or a place in a facility such as a plastic house. The place information may be manually input to the first terminal 1 by the first user, or may be specified by the first position information. Diagnosis information and the disorder DB 321 may include place information. The data set creation unit 312 may create a data set by dividing the data set for each place information (that is, whether it is an open field or a place in a facility), and the training unit 313 may create a plurality of the prediction models 341 for each place information. In this case, the second terminal 4 transmits, to the prediction server 3, place information which is specified by the manual input of the second user or by the second position information, the place information being desired to be predicted by the second user, in advance or at a transmission timing of the second position information. The information acquisition unit 314 acquires the place information from the second terminal 4 and outputs the place information to the prediction unit 315. The prediction unit 315 predicts the possibility of occurrence of the disorder by using the prediction model 341 corresponding to the place information. In general, in open-field cultivation and facility cultivation, the types of disorder occurring on the plant are different from each other. According to the above-described processes, the different prediction models 341 for the case of open-field cultivation and the case of facility cultivation are created respectively, and the possibility of occurrence of the disorder can be predicted by the prediction model 341 corresponding to the cultivation place desired by the second user. Therefore, it is possible to more accurately predict the possibility of occurrence of the disorder.

### [Implementation Example by Software]

Each control block of the control unit 11 of the first terminal 1, the control unit 21 of the diagnosis server 2, the control unit 31 of the prediction server 3, and the control unit 41 of the second terminal 4 may be implemented by a logic circuit (hardware) formed on an integrated circuit (IC chip) or the like, or may be implemented by software. In the latter case, the control unit 11, the control unit 21, the control unit 31, and the control unit 41 include a computer that executes a command of a program that is software for implementing each function. This computer includes, for example, one or more processors and a computer-readable recording medium storing the program. In the computer, the processor reads the program from the recording medium and executes the program, and thus the object of the present invention is achieved. As the processor, for example, a central processing unit (CPU) can be used. As the recording medium, a “non-transitory tangible medium”, for example, a tape, a disk, a card, a semiconductor memory, a programmable logic circuit, and the like can be used in addition to a read only memory (ROM) and the like. Furthermore, a random access memory (RAM) or the like for developing the program may be further provided. Furthermore, the program may be supplied to the computer via any transmission medium (communication network, broadcast wave, or the like) capable of transmitting the program. Note that an aspect of the present invention also can be realized in a form of a data signal embedded in a carrier wave in which the program is implemented by electronic transmission.

The present invention is not limited to the above-described embodiments, and various modifications can be made within the scope of the claims, and embodiments obtained by appropriately combining technical means disclosed in different embodiments are also included in the technical scope of the present invention.

1. A prediction device comprising:
a data acquisition unit that acquires, from a diagnosis device that diagnoses a disorder occurring on a plant, first position information indicating a growth area of the plant, a diagnosis date, and a type of the disorder; and
a training unit that constructs a prediction model predicting a possibility of occurrence of the disorder in an arbitrary area and on an arbitrary date by training a learning model about a correlation among an area indicated by the first position information, a date indicated by the diagnosis date, and a type of the disorder in a machine learning manner and,
wherein the type of the disorder is a diagnosis result estimated by the diagnosis device based on a diseased portion image of the plant on which the disorder occurs by using a diagnosis model trained about a correlation between diseased portion images of the plant on which the disorder occurs and the type of the disorder in the machine learning manner.

2. The prediction device according to claim 1, wherein the data acquisition unit acquires first environment information that is information related to a growth environment of the plant, and
the training unit trains the learning model about a correlation of the type of the disorder with the area indicated by the first position information, the date indicated by the diagnosis date and the first environment information in the area and on the date in the machine learning manner, and constructs the prediction model that predicts the possibility of occurrence of the disorder on an arbitrary date, in an arbitrary area and an arbitrary growth environment.

3. A prediction device comprising:
an information acquisition unit that acquires, from a terminal device, second position information indicating a position of the terminal device; and
a prediction unit that predicts a possibility of occurrence of a disorder in one or more areas including an area indicated by the second position information on an arbitrary date by using a prediction model trained about a correlation among an area, a date, a type of disorder occurring on a plant in a machine learning manner; and
a notification unit that notifies the terminal device of at least a prediction result of the prediction unit for the area indicated by the second position information,
wherein the type of the disorder is a diagnosis result estimated by a diagnosis device that diagnoses a disorder based on a diseased portion image of the plant on which the disorder occurs by using a diagnosis model trained about a correlation between diseased portion images of the plant on which the disorder occurs and the disorder in the machine learning manner.

4. The prediction device according to claim 3, wherein the prediction unit periodically predicts the possibility of occurrence of the disorder by using the prediction model, and
the notification unit notifies the terminal device of the prediction result in a case where the prediction result for the area indicated by the second position information satisfies a predetermined condition.

5. The prediction device according to claim 3, wherein the information acquisition unit acquires second environment information that is information related to a growth environment of the plant in each area in which the possibility of occurrence of the disorder is predictable, and
the prediction unit predicts the possibility of occurrence of the disorder based on a predetermined date, information indicating a predetermined area, and environment information in each area by using the prediction model trained about a correlation among an area, a date, the growth environment in the area and on the date, and the type of the disorder in a machine learning manner.

6. The prediction device according to claim 3, wherein the information acquisition unit acquires disorder countermeasure history data associated with an execution date of a countermeasure against the disorder and a type of the countermeasure,
the prediction device further comprises a correction unit that corrects the prediction result by using a value of a countermeasure effect obtained by inputting the execution date to a model formula for calculating the value of the countermeasure effect on and after a date when a disorder countermeasure is taken, and
the notification unit notifies the terminal device of the prediction result as corrected.

7. The prediction device according to claim 4, wherein the prediction unit corrects the prediction result according to times of occurrence of each disorder during a predetermined period in the area indicated by the second position information, and
the notification unit notifies the terminal device of the corrected prediction result.

8. The prediction device according to claim 4, wherein the information acquisition unit acquires second environment information that is information related to a growth environment of the plant in each area in which the possibility of occurrence of the disorder is predictable, and
the prediction unit predicts the possibility of occurrence of the disorder based on a predetermined date, information indicating a predetermined area, and environment information in each area by using the prediction model trained about a correlation among an area, a date, the growth environment in the area and on the date, and the type of the disorder in a machine learning manner.

9. The prediction device according to claim 4, wherein the information acquisition unit acquires disorder countermeasure history data associated with an execution date of a countermeasure against the disorder and a type of the countermeasure,
the prediction device further comprises a correction unit that corrects the prediction result by using a value of a countermeasure effect obtained by inputting the execution date to a model formula for calculating the value of the countermeasure effect on and after a date when a disorder countermeasure is taken, and
the notification unit notifies the terminal device of the prediction result as corrected.

10. The prediction device according to claim 5, wherein the information acquisition unit acquires disorder countermeasure history data associated with an execution date of a countermeasure against the disorder and a type of the countermeasure,
the prediction device further comprises a correction unit that corrects the prediction result by using a value of a countermeasure effect obtained by inputting the execution date to a model formula for calculating the value of the countermeasure effect on and after a date when a disorder countermeasure is taken, and
the notification unit notifies the terminal device of the prediction result as corrected.

11. The prediction device according to claim 8, wherein the information acquisition unit acquires disorder countermeasure history data associated with an execution date of a countermeasure against the disorder and a type of the countermeasure,
the prediction device further comprises a correction unit that corrects the prediction result by using a value of a countermeasure effect obtained by inputting the execution date to a model formula for calculating the value of the countermeasure effect on and after a date when a disorder countermeasure is taken, and
the notification unit notifies the terminal device of the prediction result as corrected.

12. The prediction device according to claim 5, wherein the prediction unit corrects the prediction result according to times of occurrence of each disorder during a predetermined period in the area indicated by the second position information, and
the notification unit notifies the terminal device of the corrected prediction result.

13. The prediction device according to claim 6, wherein the prediction unit corrects the prediction result according to times of occurrence of each disorder during a predetermined period in the area indicated by the second position information, and
the notification unit notifies the terminal device of the corrected prediction result.

14. The prediction device according to claim 8, wherein the prediction unit corrects the prediction result according to times of occurrence of each disorder during a predetermined period in the area indicated by the second position information, and
the notification unit notifies the terminal device of the corrected prediction result.

15. The prediction device according to claim 9, wherein the prediction unit corrects the prediction result according to times of occurrence of each disorder during a predetermined period in the area indicated by the second position information, and
the notification unit notifies the terminal device of the corrected prediction result.

16. The prediction device according to claim 10, wherein the prediction unit corrects the prediction result according to times of occurrence of each disorder during a predetermined period in the area indicated by the second position information, and
the notification unit notifies the terminal device of the corrected prediction result.

17. The prediction device according to claim 11, wherein the prediction unit corrects the prediction result according to times of occurrence of each disorder during a predetermined period in the area indicated by the second position information, and
the notification unit notifies the terminal device of the corrected prediction result.
